(19) United States
(12) Patent Application Publication
(10) Pub. No.: US 2021/0063390 A1
(43) Pub. Date: Mar. 4, 2021

(54) QUANTITATIVE ANALYTE DETECTION IN LATERAL FLOW IMMUNOCHEMISTRY

(71) Applicant: MIREYA C. ARONOWITZ, DELRAY BEACH, FL (US)

(72) Inventor: JACK L. ARONOWITZ, DELRAY BEACH, FL (US)

(21) Appl. No.: 17/005,635

(22) Filed: Aug. 28, 2020

Related U.S. Application Data
(60) Provisional application No. 62/893,235, filed on Aug. 29, 2019.

(51) Int. Cl. G01N 33/543

(52) U.S. Cl. CPC G01N 33/54386

(57) ABSTRACT

Lateral flow immunochemistry testing systems and methods are provided. A system can include a tester substrate with at least two control lines and at least one test sample line. A test sample with an unknown amount of analyte can be deposited on the tester substrate and the test sample can move along the tester substrate, to contact the at least two control lines and the at least one test sample line. A measuring device can be used to compare the at least one test sample line to the at least two control lines to give a quantitative value of the amount of analyte present in the test sample.

### CROSS-REFERENCE TO A RELATED APPLICATION

This application claims the benefit of U.S. Provisional Application Ser. No. 62/893,235, filed Aug. 29, 2019, which is hereby incorporated by reference herein in its entirety, including any figures, tables, and drawings.

### BACKGROUND

Lateral flow immunochemistry has been around for almost 40 years and is widely used for both point of care testing, rapid testing at home, and in clinical environments. One of the first practical uses was the one-step pregnancy test (see, e.g., U.S. Pat. No. 4,774,192). Because of the limitations of the chromatography, flow, and materials, this technique is limited in the related art to reporting simply the presence or absence of a protein or other analyte. When visually read, the best that can be done is comparison to a control line (e.g., luteinizing hormone (LH)) to determine if the sample appears stronger or weaker than the control line. With the improvements in camera technology, readers based on pixel and/or camera technology have been developed, but such readers are extremely inconsistent and expensive.

Existing lateral flow immunochemistry testers use one control and compare the sample to the control, regardless of the type of technology used in the tester. Some examples of such testing methods can be found in U.S. Pat. No. 6,528,323 (Thayer et al.), U.S. Pat. No. 8,354,270 (Polito et al.), U.S. Pat. No. 9,207,241 (Lambotte et al.), U.S. Pat. No. 9,557,329 (Lee), and U.S. Patent Application Publication No. 2003/0119203 (Wei et al.). Such testers and testing methods compare a sample to the control line to determine the presence of an analyte (e.g., if sample line is lighter than standard line=not present, else=present).

### BRIEF SUMMARY

In view of the limitations of related art lateral flow immunochemistry testers and testing methods, there exists a need in the art for an improved tester/reader and testing method for lateral flow immunochemistry.

Embodiments of the subject invention provide novel and lateral flow immunochemistry testing systems and methods that address the disadvantages and limitations of related art systems and methods. A system can include a tester substrate with at least two control (known-value) zones that can each produce a control indicator line corresponding to a known value of an analyte, one having a lower known value than the other. The analyte can be conjugated to marker that can be measured with a measuring device. There can also be at least one test sample zone that can produce a test sample line when contacted with the conjugated-analyte in the test sample. A test sample with an unknown amount of conjugated-analyte can be deposited on a tester substrate, such as, for example, in a sample well that is in contact with the tester substrate. The test sample can migrate by capillary action, through the tester substrate, until it reaches the at least two control zones and the at least one test sample zone, whereby at least two control lines are created and at least one test sample line is created. A reader, meter, or other measuring device can be used to compare the at least one test sample line to the at least two known-value control lines, which are used as or can provide a standard against which to compare the test sample line to quantify the amount of analyte present in the test sample. The embodiments of the systems and methods of the subject invention are inexpensive and provide accurate and repeatable results.

### BRIEF DESCRIPTION OF THE DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1 shows one embodiment of a test strip with two control zones, where one control zone has a higher analyte-binding value than the other control zone, and a test sample zone between the control zones.

FIGS. 2A and 2B show an embodiment of a cassette having three separate viewing windows in which a tester substrate can be secured. FIG. 2A shows the cover of the cassette and FIG. 2B shows the base of the cassette that can be connected to the cover.

FIGS. 3A and 3B show alternative views of the cassette in FIGS. 2A and 2B. FIG. 3A shows the interior of the cover and FIG. 3B shows the interior of the base.

FIGS. 4A and 4B show an alternative embodiment of a cassette having a single viewing window. FIG. 4A shows a top plan view with a well for receiving a sample and the viewing window. FIG. 4B shows a sample being deposited in the well of the cassette.

FIG. 5 shows a representative non-limiting example of a measuring device that can be used to measure the analyte bound to the two or more control zones and the one or more test sample zones.

FIGS. 6A, 6B, 6C, and 6D illustrate an alternative embodiment of a cassette in which an embodiment of a test strip can be secured. FIG. 6A is a perspective view of the cassette ready for use. FIG. 6B is a side elevation view of a cross-section of the cassette. FIG. 6C shows a top plan view of the interior of the cover of the cassette with an embodiment of a test strip. FIG. 6D shows the interior of the base of the cassette, which can be connected to the cover, with an embodiment of the test strip.

### DETAILED DESCRIPTION

Embodiments of the subject invention provide novel lateral flow immunochemistry testing systems and methods that address the disadvantages of related art systems and methods. In lateral flow immunochemistry tests, where color and/or intensity is used as an indicator, it can be difficult to reproduce results across different tests because the color can be slightly lighter or slightly darker or intensity can vary between tests. Thus, tests in the related art typically provide binary results, indicating the presence or absence of an analyte, but do not provide quantitative results. It has previously been especially difficult to produce repeatable, consistent results when using colloidal gold as the color and/or intensity marker for an analyte, which is the most common color and/or intensity marker utilized in lateral flow chemistry tests. Embodiments of the subject invention address this challenge by using standard or known-value control lines for comparison with a test sample line.

In the description that follows, a number of terms are used related to embodiments of the subject invention. In order to provide a clear and consistent understanding of the specification and claims, including the scope to be given such terms, the following definitions are provided.

As used herein, the terms “proximal end” or “proximal direction” refer to that end closest to or near an opening or “well” in which a test sample can be deposited on a tester substrate.

As used herein the terms “distal end” or “distal direction” refer to that end closest to the absorber or that end furthest from where a test sample is deposited on a test strip.

When the term “approximately” or “about” is used herein, in conjunction with a numerical value, it is understood that the value can be in a range of 95% of the value to 105% of the value, i.e. the value can be +/−5% of the stated value. For example, “about 1 kg” means from 0.95 kg to 1.05.

Embodiments of the subject invention use can include a tester substrate 100 on which a test sample 160 can be absorbed or taken in to migrate, flow, or otherwise move across or through the tester substrate. The tester substrate can have at least two control zones, where an analyte-binding value in each control zone is known to a high degree of certainty. A Low control zone 110 can have a first known analyte-binding value (e.g., 20 nanograms per milliliter (ng/ml)) and a High control zone 120 can have a known analyte-binding value different from and higher than the Low control zone analyte-binding value (e.g., 100 ng/ml). Though it is not necessary, the first and second known analyte-binding values can be chosen such that any test sample is expected (or likely, or very likely) to provide a result that is between the High and Low known analyte-binding values. A test sample deposited on the tester substrate can migrate or move through the tester substrate so that it encounters the Low control zone, High control zone and the test sample zone.

In an embodiment, a system can include a reader, meter or other measuring device 300 and a tester substrate 100 (e.g., a test strip, test cassette, or similar test substrate). The test substrate can include at least three zones that result in at least three lines. In one embodiment,
one line is a Low control zone 110 in which a Low control line 115 can be formed and used to establish a “low” standard (based on the lower known analyte-binding value), one is a test sample zone 160 for the test sample that produces a test sample line 135 based on the amount of conjugated-analyte 160, with an associated marker, bound thereto, that is used to obtain a test sample value 136 and one is for the High control zone 120 in which a high control line 125 is formed and used to establish the “high” standard (based on the higher known analyte-binding value). The three zones and resulting lines formed therein by contact with the conjugated-analyte can be parallel or approximately parallel, though embodiments are not limited thereto. The high and low standards can each be determined by control zones that contain or have a predetermined, known, specific amount of analyte-binding substance 105, such as, for example, an antibody or antigen, such that the desired quantity of conjugated-analyte, such as, for example a conjugated-antigen or conjugated-antibody, will bind to the analyte-binding substance. In one embodiment, the ratio of the analyte-binding substance to the conjugated-analyte that can be bound thereto is about 1:1. For example, there is typically a 1:1 ratio of conjugated-antibody:binding antigen or conjugated-antigen:binding antibody. Of course, other ratios could be used (e.g., 2:1, 1:2, 3:1, 1:3, or any other ratio, including non-whole-number ratios).

In a further embodiment, the test sample zone 130 can have an amount of analyte-binding substance 105, such as, for example, an antibody or antigen, that is effectively “unlimited” in the amount of conjugated-analyte 160 that can be bound thereto, by having an analyte-binding value that is higher than would be required to measure the analyte in any reasonably expected sample. If the analyte to be tested is an antigen, then the zones can each have antibody thereon, and if the analyte to be tested is an antibody, then the zones can each have antigen thereon.

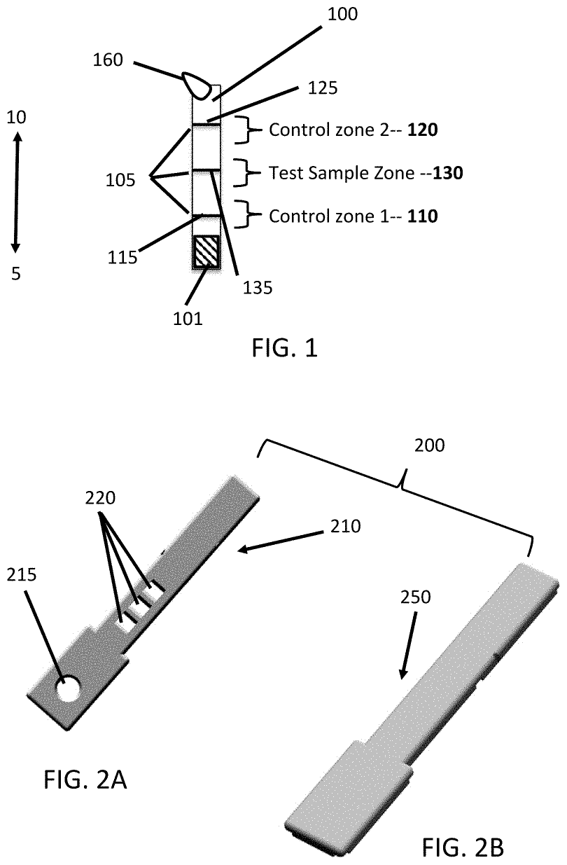

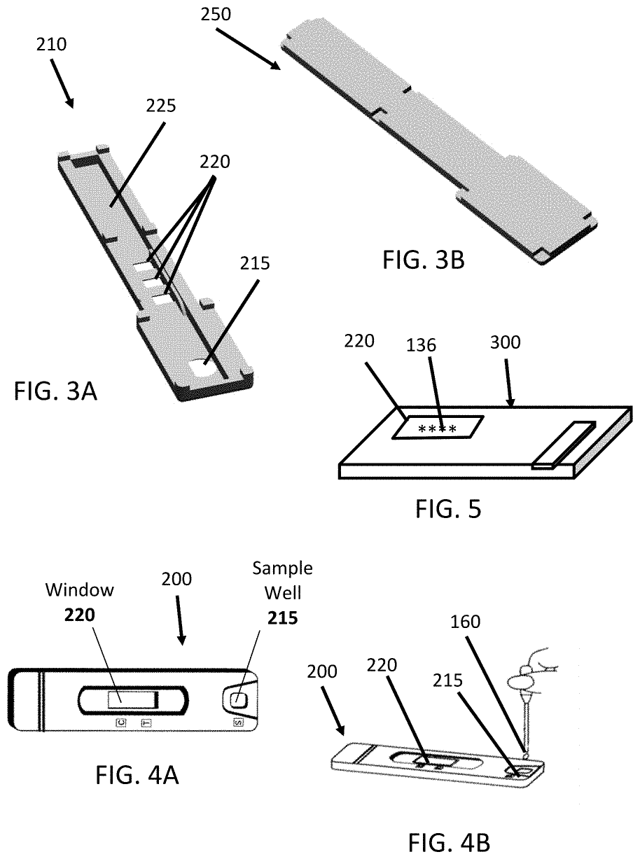

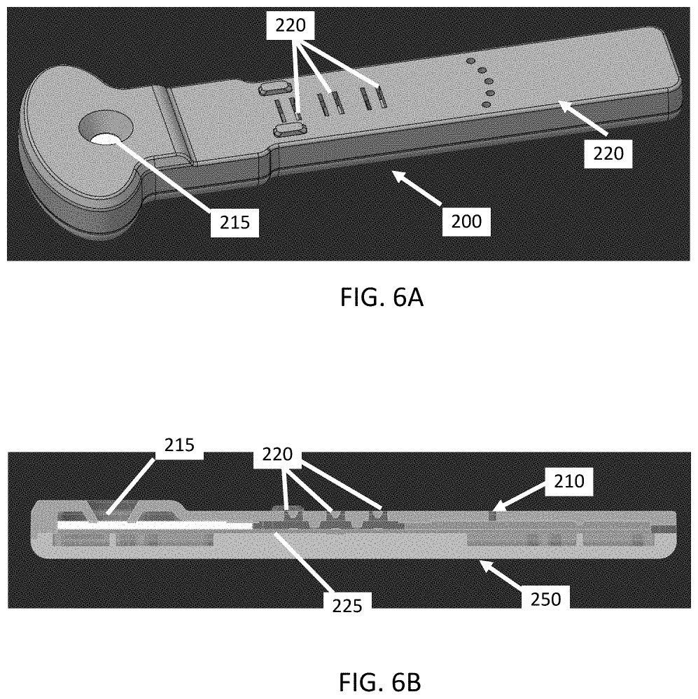

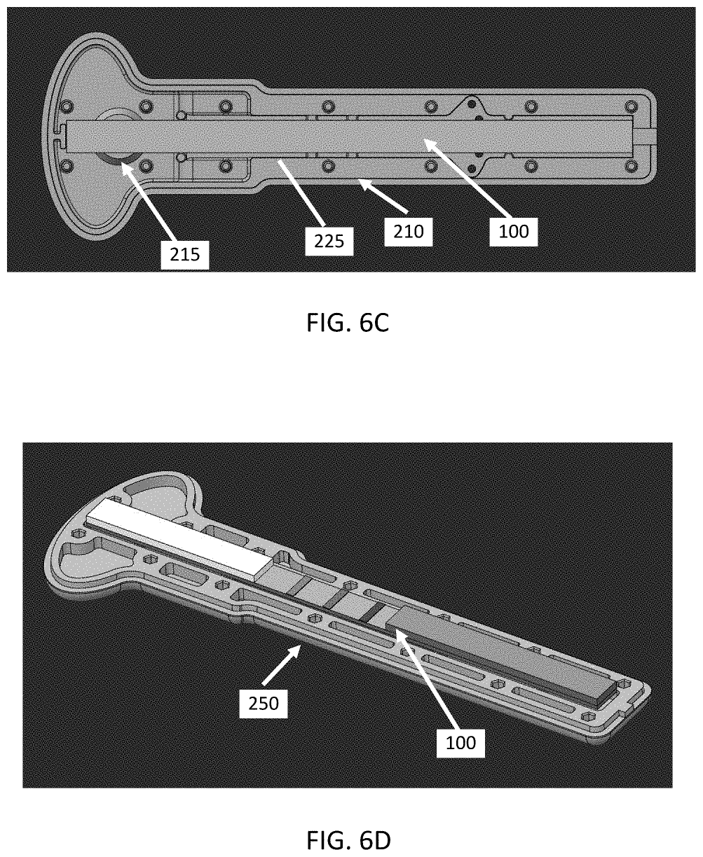

In one embodiment, the testing system can comprise a cassette 200 that can at least include a tester substrate 100 on which the various zones are disposed, such as shown, for example, in FIG. 1, within a base 250 (which can be shaped to form a case) in which the tester substrate can be secured, as shown, for example, in FIGS. 2A, 2B, 3A,3B, 6C, and 6D. In one embodiment, the case secures the tester substrate 100 in a precise location therein to facilitate accurate measurements of the Low control line 115, the High control line 125, and the test sample line 135 formed within the Low control zone 110, the High control zone 120, and the test sample zone 130, respectively. One example of a cover 210 and a base 250 is shown in FIGS. 2A and 2B. FIGS. 6C and 6D show a cover 210 and base 250 for an alternative embodiment of a cassette. The cover can have an opening or well 215 at a proximal end 5 in which the test sample can be disposed, and the system is configured such that the well permits direct contact with the tester substrate 100, such that the test sample therein, when present directly contacts the tester substrate. In one embodiment, the cover has an internal seat or slot 225 in which the tester substrate 100 can be fittingly seated to inhibit movement in the cassette. An example of the internal slot in a case is shown in FIGS. 3A and 6C. The cover can also have one or more windows 220 through which the control zones and test sample zone can be seen or accessed by a measuring device 300. FIGS. 2A, 3A, and 6A show embodiments of a cassette 200 with three windows. FIGS. 4A and 4B illustrate an alternative embodiment of a cassette having a single window. The base 250 can attach to the cover 210 to enclose and secure the tester substrate in place in the internal slot. In a specific embodiment, the cover 210 can snap together with a base. In further specific embodiment, the cassette can be cooperatively engaged with a measuring device 300 to position the one or more windows in a proper alignment for the measuring device to access and measure or read a High control line 120, Low control line 115, and a Test sample line 135 thereon.

The system can further include an absorber 101 at the distal end and in contact with the tester substrate 100. In one embodiment, the absorber is part of or incorporated as part of the tester substrate. In another embodiment, the absorber is a separate element from the tester substrate. The absorber 101 can be in direct physical contact with the tester substrate. An absorber can comprise any material, substance, or device that can take up, absorb, draw in, soak in, or otherwise “drive” or force a test sample 160 to move across or through a tester substrate. When a test sample 160 is deposited in the well 215, capillary action causes the test sample to be absorbed initially by the tester substrate 100 so that it migrates across the tester substrate until it reaches the absorber. The absorber can take up or absorb the test sample migrating to towards the distal end 10 of the tester substrate, thereby driving the capillary flow across the tester substrate and promoting the movement of the test sample across the tester substrate in a proximal 5 to distal 10 direction.

In one embodiment, the system can be configured such that the test sample 160 contacts the Low control zone with the low analyte-binding value, to create a Low control line 115 that can be measured to determine a “low” standard, then it contacts the test sample zone 130 where it creates a test sample line that can be measured to obtain a test sample value 136, and then finally the test sample contacts High control zone with the high analyte-binding value to create the “high” standard line that can be measured to determine the “high” standard. In an alternative embodiment, the system can be reversed and configured such that the test sample first contacts the High control zone with the high analyte-binding value to create the “high” standard line, then it contacts the test sample zone wherein it creates a test sample line, and then finally it contacts the Low control zone with the low analyte-binding value to create the “low” standard line. This reverse configuration can be advantageous with competitive analyte-binding substances, where the reaction causes the color and/or intensity to get weaker or is less pronounced as the amount of analyte being bound increases. In general, the standard line that is expected to have the lighter color and/or less intensity during/after testing can be closest to the well 215 and the other standard line, expected to have the darker color and/or higher intensity during/after testing, can be closest to the absorber 101. If the analyte test is such that color and/or intensity increases with quantity of analyte present, then the low standard line can be closest to the well, alternatively, if the analyte test is such that color and/or intensity decreases with quantity of analyte present (e.g., as with competitive binding), then the high standard line can be closest to the well.

The system can also include a reading or measuring device 300. After the test sample is placed on the testers substrates, such as, for example, in the well, and has contacted the Low control zone, Test sample zone, and, possibly, after the test sample has reached the high standard line, the tester substrate 100, either alone or as a cassette 200, can be examined with the measuring device, for example, the tester substrate can be placed in or otherwise cooperatively engaged with the measuring device (e.g., a reflectance measuring device), which can calculate a reflectance value, a.k.a., an analyte-binding value, for each control line and the test sample line 135. The measuring device can be programmed to have a waiting period (e.g., 5-15 minutes, or 10-15 minutes, or 10-20 minutes), or a waiting period between any of the two listed values. The waiting period can allow the color and/or intensity development of the control and test sample lines to stop changing and to stabilize. After the pre-determined waiting period, the measuring device can then determine a quantitative amount of the analyte present in the test sample line by comparing with the “High” standard line and the “Low” standard lines relative to an extrapolation curve.

Comparison of the test sample line with the “High” and “Low” standard lines can be done using a first algorithm, which can be run by a processor (e.g., a microprocessor) of the reading device (the first algorithm can be stored (e.g., as code) on a storage medium (e.g., (non-transitory) machine-readable medium) of the reading device). The processor and/or storage medium can instead be on an external device (that can be considered part of the system or can be considered separate from the system) that is in operable communication with the reading device while it is determining the amount of analyte present. The measuring device 300 can be calibrated by obtaining measurements from multiple analyte samples of varying concentrations that are each known to a very high degree of accuracy. A second algorithm, which can be the same as or different from the first algorithm), is used to determine an extrapolation curve. In one embodiment, the extrapolation curve is a straight line, representing a linear relationship between the “High” standard and the “Low” standard and their known analyte-binding values. In an alternative embodiment, the extrapolation curve is a curve, representing a non-linear relationship between the “High” standard and the “Low” standard. The extrapolation curve can be used, in combination with the first algorithm to determine a quantitative amount of analyte that was present in the test sample. Alternatively, the extrapolation curve can be used without the first algorithm, or in combination with a third algorithm that may be the same as or different from the second algorithm, to determine a quantitative amount of analyte that was present in the test sample.

Advantageously, variations between the “High” and/or “Low” standard lines between different tester substrates can be accounted and/or adjusted for, therefore variations between tester substrates can be inhibited from affecting the accuracy of the quantitative determination of the amount of analyte present in a particular test sample value. This is the case because the relationship between colors and/or intensities of the known “High” and known “Low” standard lines will remain the same and can be fit or compared to the extrapolation curve, thereby allowing the test sample value to also be fit to or compared to the extrapolation curve. Thus, for any given set of measurements the extrapolation curve will be the same and the measurements will still be repeatable and accurate.

In one embodiment, an extrapolation curve for use in calculating a test sample value 136 for a test sample line 135 with a measuring device 300 can be obtained using an algorithm (e.g., such as, for example, the second algorithm mentioned above) and calibration with multiple (e.g., 2, 3, 4, 5, or more) known quantities. For example, a straight-line extrapolation can be assumed until a certain level (high or low), at which point there can be a 10% reduction or decrease in slope (or another particular increase in slope) or change to a curve of a certain inflection (second derivative). Then, at another level the curve changes again, and at another level (if more than two known quantities are used) it can be changed again, and this can be repeated as desired. A person of skill in the art will be able to determine an appropriate method for calculation of an extrapolation curve.

In an embodiment, a measuring device 300 is a disposable reflectance meter. The term “disposable” is used in its normal context, meaning the measuring device is intended to be used only once or for at a short period of time (e.g., just with the provided test substrates that come with the meter) and is made using inexpensive and disposable materials.

A processor of the disposable reflectance meter can be calibrated in advance using an algorithm, such as, for example, the second algorithm discussed above, and an extrapolation curve. This data can be stored, for example, as software or code, on a storage medium of the meter. Further, the disposable reflectance meter can be part of a system or kit that includes a set or pre-determined number of tester substrates, as test strips or cassettes, (e.g., 10, 15, 20, 25, 30, 40, 50, 60, 70, 80, 90, 100, 200, etc.). The reflectance meter and can be calibrated for those tester substrates that are provided with the reflectance meter. In one embodiment, a different disposable reflectance meter can be specifically calibrated for a specific “lot” of tester substrates (i.e., each kit can include a disposable reflectance meter and a set amount of tester substrates, the disposable reflectance meter being specifically calibrated for those tester substrates). The disposable reflectance meter can be disposed of after using the test substrates included in the kit.

In certain embodiments, in addition to an algorithm for calculating a test sample value 136 for the test sample line 135 based on readings of the high control line 125 and the low control lines 110, a measuring device 300 (e.g., disposable reflectance meter) can also be pre-programmed with a look-up table to correct for variations in dose response, as the levels get higher or lower. This can be based on, for example, a curve, or compound curve, versus a straight line, for increasing or decreasing amounts of analyte.

While colorimetry and spectrophotometry absorbance technology has been used for other types of testing (e.g., glucose testing), it has not been applied in the related art to methods of lateral flow antibody/antigen immunochemistry. Application of colorimetry and spectrophotometry absorbance to lateral flow antibody/antigen immunochemistry has not previously been thought possible, in addition to other reasons, because of the color variation that occurs on the measuring devices.

Embodiments of the subject invention address this challenge by using two control lines that establish a “High” standard and “Low” standard, in addition to the test sample line 135, on the same tester substrate 100, which can all be measured simultaneously. An algorithm and/or extrapolation curve can be used to compare the measurements therewith and increase/ensure accuracy and repeatability between tests. In one particular embodiment, the measuring device has been specifically calibrated in advance for use with a specific group of tester substrates as part of a kit.

Analyte testing using embodiments of the systems and methods of the subject invention can be based on regular binding (where a darker color indicates a higher amount of analyte) or competitive binding (where a lighter color indicates a higher amount of analyte). Because of the ability to produce a measured response in control lines with respective known values of an analyte, embodiments of the subject invention use at least two control lines (e.g., exactly two control lines). The control lines advantageously provide standards with a measurable response representative of a specific known amount of analyte. The testing line (and/or testing zone) need not be between the control lines (and/or control zones), though it can be in some embodiments. Ideally, the response can be measurable with a simple, cheap, and accurate measuring device. The measuring device, such as, for example, a meter/reader can be based on reflectance photometry, though embodiments are not limited thereto. The technique of using at least two control lines as discussed herein can be effective if the marker conjugated or bound to the analyte (or other moiety) is visual (e.g., colloidal gold, latex), magnetic, fluorescent, or any other means suitable for identifying the results of binding of the analyte involved, for example, if the analyte is an antigen or an antibody. The marker can bind to a moiety that is the analyte itself or is bound to the analyte (e.g., a conjugate or part of a conjugate), as long as the color can be compared. Though the term “analyte-binding” is used herein, in each instance, this could be “moiety-binding” where the moiety is the analyte itself or is bound to the analyte (e.g., a conjugate or part of a conjugate).

By producing at least two control lines with known moiety-binding values on the same tester substrate as the test sample line, and measuring the results produced by these lines at a stable point in the reaction (or as a rate of reaction, or by following a plot intercept), two known readings are produced and the quantitative amount of analyte in the test sample can then be accurately determined. It is preferred, though not necessary, that the quantitative amount of analyte in the test sample is between the respective amounts of analyte in the “Low” 110 and “High” 120 control zones. In other words, the measureable amount of the conjugated-analyte 160 is preferably between the High standard and the Low standard derived from the “High” and “Low” control lines.

When the calculated “standard” or “extrapolation” curve is produced (e.g., by the meter or reading device), it is possible to extend the calibrations above and/or below the values for the standards to accurately plot values both smaller than that of the Low standard and/or higher than that of the High standard. This is particularly accurate if the High and Low standards produce a straight-line calibration. Even if, however, the High and Low standards do not produce a straight-line calibration, the measuring device (reader or meter) can be pre-calibrated for use with a particular set, lot, or group of tester substrates 100. In one embodiment, the measuring device can be calibrated by pre-testing with a plurality of samples of analyte-binding substance with known values, to produce an accurate non-linear calibration curve, such that standard values higher than the “High” control line and/or lower than the “Low” control line can be predicted with good accuracy from a programmed calibration curve.

The same basic criteria of spectrophotometry or colorimetry can be used for the visual readings (e.g., the color intensity of the test sample is to the quantitative value of analyte in the test sample as the color intensity of the low and/or high standard is to the value(s) of said standard(s)), though embodiments are not necessarily limited thereto). Similarly with any magnetic, fluorescent, or other type of measurement (including visual), a standard curve (can be referred to an extrapolation curve) can be generated that can allow a measuring device (meter/reader) to be calibrated automatically during the actual test. Further, because the test sample line is generated on same medium or tester substrate 100 as the High and Low control lines, it can consistently produce an accurate result based on the two or more control line measurements or readings that are performed simultaneously and using the same chemistry at the same time (using the same meter/reader). In other words, the variations between tester substrates 100 can be adjusted for by the embodiments of the subject invention by providing high and low controls unique to each tester substrate that can be compared to an extrapolation curve to calculate an accurate test sample value obtained from the same tester substrate (e.g., the conjugate).

Existing lateral flow immunochemistry testers and testing methods usually cannot produce accurate and repeatable results without using enzyme-linked immunosorbent assay (ELISA) or polymerase chain reaction (PCR), both of which are laborious, time-consuming, and very expensive. Embodiments of the subject invention provide accurate, repeatable, quantitative results using a quick and inexpensive system/method.

Though testing based on color change is discussed at length herein, embodiments of the subject invention can be used instead for testing based on magnetic, fluorescent, or any other means suitable for identifying the results of binding of the analyte involved (whether the analyte is an antigen or an antibody). The same principles apply, though the reading device/meter would need to be configured to read/analyze the appropriate property (magnetic, fluorescent, etc.).

Some embodiments of the subject invention may have some aspects in common with the devices disclosed in U.S. Pat. No. 6,574,425 (Weiss et al.) and U.S. Pat. No. 6,952,263 (Weiss et al.), both of which are hereby incorporated by reference herein in their entireties.

The subject invention includes, but is not limited to, the following exemplified embodiments.

### Embodiment 1

A tester substrate, configured to obtain a quantitative amount of an analyte in a test sample, the tester substrate comprising:

at least one test sample zone comprising a moiety-binding substance that binds to a moiety that is the analyte or is bound to the analyte; and

at least two control zones, each control zone comprising a known different amount of the moiety-binding substance, and configured such that when contacted with the analyte in the test sample, binds with a corresponding known amount of the moiety in the test sample, such that obtaining a measure of the amount of moiety bound to each control zone provides standards against which a measure obtained for the amount of moiety bound to the test sample zone can be compared to determine a quantitative amount of the analyte in the test sample.

### Embodiment 2

The tester substrate according to embodiment 1, wherein the moiety is conjugated to a visual, magnetic, or fluorescent marker that causes a test sample line to be formed when the moiety binds to the at the at least one test sample zone and causes a control line to be formed in each of the at least two control zones.

### Embodiment 3

The tester substrate according to any of embodiments 1-2, wherein the line is measured to obtain the quantitative amount for the analyte bound at each of the at least one test zone and the at least two control zones.

### Embodiment 4

The tester substrate according to any of embodiments 2-3, further comprising a 1:1 ratio between the marker and the analyte.

### Embodiment 5

The tester substrate according to any of embodiments 2-4, wherein the measuring device is configured to utilize colorimetry or spectrophotometry techniques to measure the marker.

### Embodiment 6

The tester substrate according to any of embodiments 1-5, wherein the analyte in the test sample first contacts a control zone, of the at least two control zones, having the higher amount of moiety-binding substance.

### Embodiment 7

The tester substrate according to any of embodiments 2-6, wherein the amount of marker present in each of the at least one test sample zone and the at least two control zones is measured to obtain the quantitative amount of moiety bound at each of the at least one test zone and the at least two control zones.

### Embodiment 8

A kit for obtaining a quantitative amount of an analyte in a test sample, the kit comprising:

at least one tester substrate having:
at least one test sample zone comprising an moiety-binding substance; and
at least two control zones each comprising known different amounts of the moiety-binding substance,
such that, when the analyte contacts the moiety-binding substance in the at least one test sample zone a test sample line is formed and, when the analyte contacts the moiety-binding substance in the at least two control zones and a control zone line is formed in each; and

a measuring device, having access to a calibration curve, that measures each of the control zone lines and compares the measurements with the calibration curve to determine a high standard and a low standard on the calibration curve that correspond to the known amounts of moiety-binding substance present in each of the respective control zones and that measures the test sample line and compares the test sample line to the high standard and the low standard to determine the amount of moiety bound to the moiety-binding substance in the test sample zone.

### Embodiment 9

The kit according to embodiment 8, further comprising a pre-determined number of tester substrates,

wherein the measuring device is configured to measure the pre-determined number of tester substrates.

### Embodiment 10

The kit according to any of embodiments 8-9, wherein each of the tester substrates is secured in a cassette.

### Embodiment 11

The kit according to embodiment 10, wherein the cassette is cooperatively engaged with the measuring device.

### Embodiment 12

The kit according to any of embodiments 8-11, wherein the analyte is conjugated to a visual, magnetic, or fluorescent marker.

### Embodiment 13

A method for determining an amount of analyte in a test sample comprising:

depositing the test sample onto the tester substrate according to any of embodiments 1-7 (or the at least one tester substrate of the kit according to any of embodiments 8-12);

allowing the test sample to contact a first of the at least two control zones to form a first control line;

allowing the test sample to contact the at least one test sample zone to form a test sample line;

allowing the test sample to contact a second of the at least two control zones to form a second control line;

utilizing a measuring device to measure the first control line, the test sample line, and the second control line;

fitting the first control line and the second control line to a calibration curve programmed into the measuring device; and

comparing the measurement of the test sample line to the fitted first control line and the fitted second control line to determine the amount of moiety bound to the test sample zone, thereby giving the amount of the analyte in the test sample.

### Embodiment 14

The method according to embodiment 13, wherein the moiety is conjugated to a visual, magnetic or fluorescent marker that forms the test sample line, the first control line, and the second control line, and

wherein the method further comprises measuring, by the measuring device, the amount of marker present at the test sample line, the first control line, and the second control line.

### Embodiment 15

The method according to any of embodiments 13-14, wherein the tester substrate is secured in a cassette, and

wherein the method further comprises cooperatively engaging the cassette with the measuring device.

### Embodiment 16

The method according to any of embodiments 13-15, wherein the measuring device utilizes colorimetry or spectrophotometry to measure the test sample line, the first control line, and the second control line.

### Embodiment 17

The method according to any of embodiments 13-16, wherein the amount of moiety-binding substance at the first control zone is greater than the amount of moiety-binding substance at the second control zone.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to the invention.

### Example 1

A lateral flow immunochemistry tester substrate (e.g., test strip or test cassette) to test for vitamin D includes a “low” standard line and a “high” standard line, with a test sample line disposed therebetween. It is noted that testing for vitamin D is based on competitive binding, which means that (similar to many drug or small molecule tests), the color reaction gets weaker as the amount of analyte increases. The “high” standard line comprises a known amount of 100 ng/ml of vitamin D and the “low” standard line comprises a known amount of 20 ng/ml of vitamin D. The antibody trapping at the control lines is reactive to a specific amount of substance bound to the same conjugate as the antibody for vitamin D. Colloidal gold is used as the color marker, and each antibody trapping control line (which provides measurement values for a high standard, test sample, and low standard) will capture its proportional and representative amount of colored particles. By measuring the reflectance of the two control lines, the exact quantitative amount of vitamin D present in the test sample line therebetween can be calculated with clinical laboratory accuracy.

### Example 2

A disposable reflectance meter is calibrated (using an algorithm) for 20 lateral flow immunochemistry test cassettes to test for vitamin D in a sample. Each test cassette includes a low control line that, when measured, provides a Low standard value and a high control line that, when measured, provides a High standard value, with a test sample line disposed therebetween. The high control line contains a known amount of 100 ng/ml of vitamin D and the low control line is for a known amount of 20 ng/ml of vitamin D. During testing, the test cassette can be positioned such that the high control line is closer to the well of the cassette than the low control line, such that the low control line is closer to the absorber than the high control line. Each test cassette can be used to conduct one test to determine the quantitative amount of vitamin D present in a sample. The disposable reflectance meter can be disposed of after the 20 test cassettes are used.

### Example 3

A lateral flow immunochemistry tester substrate (e.g., test strip or test cassette) to test for D-dimer (a protein that determines clotting function) includes a low control line that provides a value for a low standard and a high control line that provides a value for high standard, with a test sample line disposed therebetween. It is noted that testing for D-dimer is not based on competitive binding, so the color reaction gets stronger as the amount of conjugated-analyte increases, which in this example is due to the amount of gold trapped at each line. The high standard line comprises a known amount of 300 ng/ml (alternatively could be 330 ng/ml) of D-dimer and the low standard line comprises a known amount of 100 ng/ml of D-dimer. By measuring the reflectance of the two controls, and comparing/fitting the results to an extrapolation curve, the exact quantitative amount of D-dimer present in the test sample (via measuring the test sample line) can be calculated with clinical laboratory accuracy.

### Example 4

A disposable reflectance meter is calibrated (using an algorithm) for 20 lateral flow immunochemistry test cassettes to test for D-dimer. Each test cassette includes a control line that provides a low standard and a control line that provides high standard, with a test sample line disposed therebetween. The control line for the high standard is for a known amount of 300 ng/ml (alternatively could be 330 ng/ml) of D-dimer and the control line for the low standard is for a known amount of 100 ng/ml of D-dimer. During testing, the test cassette can be positioned such that the high standard control line is closer to the absorber than the low standard control line, such that the low standard control line is closer to the well in the cassette than the high standard line. Each test cassette can conduct one test to determine the quantitative amount of D-dimer present in a sample. The disposable reflectance meter can be disposed of after the 20 test cassettes are used.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A tester substrate, configured to obtain a quantitative amount of an analyte in a test sample, the tester substrate comprising:
at least one test sample zone comprising a moiety-binding substance that binds to a moiety that is the analyte or is bound to the analyte; and
at least two control zones, each control zone comprising a known different amount of the moiety-binding substance, and configured such that when contacted with the analyte in the test sample, binds with a corresponding known amount of the moiety in the test sample, such that obtaining a measure of the amount of moiety bound to each control zone provides standards against which a measure obtained for the amount of moiety bound to the test sample zone can be compared to determine a quantitative amount of the analyte in the test sample.

2. The tester substrate according to claim 1, wherein the moiety is conjugated to a visual, magnetic, or fluorescent marker that causes a test sample line to be formed when the moiety binds to the at the at least one test sample zone and causes a control line to be formed in each of the at least two control zones.

3. The tester substrate according to claim 2, wherein the line is measured to obtain the quantitative amount for the analyte bound at each of the at least one test zone and the at least two control zones.

4. The tester substrate according to claim 3, further comprising a 1:1 ratio between the marker and the analyte.

5. The tester substrate according to claim 3, wherein the measuring device is configured to utilize colorimetry or spectrophotometry techniques to measure the marker.

6. The tester substrate according to claim 3, wherein the analyte in the test sample first contacts a control zone, of the at least two control zones, having the higher amount of moiety-binding substance.

7. The tester substrate according to claim 2, wherein the amount of marker present in each of the at least one test sample zone and the at least two control zones is measured to obtain the quantitative amount of moiety bound at each of the at least one test zone and the at least two control zones.

8. The tester substrate according to claim 7, further comprising a 1:1 ratio between the marker and analyte.

9. The tester substrate according to claim 7, wherein the measuring device is configured to utilize colorimetry or spectrophotometry techniques to measure the marker.

10. The tester substrate according to claim 7, wherein the analyte in the test sample first contacts a control zone, of the at least two control zones, having the higher amount of moiety-binding substance.

11. A kit for obtaining a quantitative amount of an analyte in a test sample, the kit comprising:
at least one tester substrate having:
at least one test sample zone comprising an moiety-binding substance; and
at least two control zones each comprising known different amounts of the moiety-binding substance,
such that, when the analyte contacts the moiety-binding substance in the at least one test sample zone a test sample line is formed and, when the analyte contacts the moiety-binding substance in the at least two control zones and a control zone line is formed in each; and
a measuring device, having access to a calibration curve, that measures each of the control zone lines and compares the measurements with the calibration curve to determine a high standard and a low standard on the calibration curve that correspond to the known amounts of moiety-binding substance present in each of the respective control zones and that measures the test sample line and compares the test sample line to the high standard and the low standard to determine the amount of moiety bound to the moiety-binding substance in the test sample zone.

12. The kit according to claim 11, comprising a pre-determined number of tester substrates,
wherein the measuring device is configured to measure the pre-determined number of tester substrates.

13. The kit according to claim 11, wherein each of the tester substrates is secured in a cassette.

14. The kit according to claim 13, wherein the cassette is cooperatively engaged with the measuring device.

15. The kit according to claim 12, wherein the analyte is conjugated to a visual, magnetic, or fluorescent marker.

16. A method for determining an amount of analyte in a test sample comprising:
depositing the test sample onto the tester substrate according to claim 1;
allowing the test sample to contact a first of the at least two control zones to form a first control line;
allowing the test sample to contact the at least one test sample zone to form a test sample line;
allowing the test sample to contact a second of the at least two control zones to form a second control line;
utilizing a measuring device to measure the first control line, the test sample line, and the second control line;
fitting the first control line and the second control line to a calibration curve programmed into the measuring device; and
comparing the measurement of the test sample line to the fitted first control line and the fitted second control line to determine the amount of moiety bound to the test sample zone, thereby giving the amount of the analyte in the test sample.

17. The method according to claim 16, wherein the moiety is conjugated to a visual, magnetic or fluorescent marker that forms the test sample line, the first control line, and the second control line, and
wherein the method further comprises measuring, by the measuring device, the amount of marker present at the test sample line, the first control line, and the second control line.

18. The method according to claim 17, wherein the tester substrate is secured in a cassette, and
wherein the method further comprises cooperatively engaging the cassette with the measuring device.

19. The method according to claim 17, wherein the measuring device utilizes colorimetry or spectrophotometry to measure the test sample line, the first control line, and the second control line.

20. The method according to claim 19, wherein the amount of moiety-binding substance at the first control zone is greater than the amount of moiety-binding substance at the second control zone.
